(19) United States
(12) Patent Application Publication
(10) Pub. No.: US 2017/0080391 A1
(43) Pub. Date: Mar. 23, 2017

(54) HIGHLY EFFICIENT REVERSE OSMOSIS FILTER

(71) Applicant: E.W. - Hydrophilic Processes Ltd., Kfar Saba (IL)

(72) Inventor: Avital Ehre, Kfar Saba (IL)

(73) Assignee: E.W. - Hydrophilic Processes Ltd., Kfar Saba (IL)

(21) Appl. No.: 15/124,575

(22) PCT Filed: Mar. 19, 2015
(86) PCT No.: PCT/IL2015/050295

§ 371 Date: Sep. 8, 2016

Related U.S. Application Data
(60) Provisional application No. 61/955,260, filed on Mar. 19, 2014.

(51) Int. Cl. B01D 69/10; B01D 61/02; C02F 1/44; B01D 71/56; B01D 71/64; C07K 5/02; B01D 71/16

(52) U.S. Cl. CPC B01D 69/105; C07K 5/0202; B01D 61/025; B01D 71/16; B01D 71/56; B01D 71/64; C02F 1/441

(57) ABSTRACT

A reverse-osmosis membrane filter comprising: a porous support layer; a porous skin layer, and at least one water binding composition predominantly bound between the skin layer and the support layer.

### FIELD OF THE INVENTION

The present invention relates to reverse osmosis membrane filters that include peptoids.

### BACKGROUND OF THE INVENTION

Filtration is a process that separates components from a fluid stream by passage of the fluid through porous medium (membrane). In membrane filtration, the membrane acts as a selective barrier that permits passage of some components (“permeate” stream) and retains others (“retentate” stream); splitting one feed-stream into two product streams. It is common to classify membranes and membrane separation processes due to size of the separated components, structure properties, driving force and mode of operation. The major membrane separation processes that are typically used in water systems are: reverse osmosis (RO), nanofiltration (NF), ultrafiltration (UF) and microfiltration (MF).

Water membrane filtration (i.e. desalination) is an active pressure-driven process. There is a need in the art of water membrane filtration to reduce the pressure (energy) that is required for the process.

Polyamide TFC membranes are currently the main type of membrane used for desalination by RO. The dense but thin active polyamide skin of the membrane is formed on top of a microporous support which is usually made of a polysulfone.

In the desalination process, external pressure motivates water passage through the skin, from the high salt concentration (salty solution), to the low salt concentration area on the support side (desalted water).

Reducing the difference in free energy between the two sides of the membrane (between the salty solution and the desalted water) would lead to a lower external pressure being required for the process, making the desalination processes more energy favorable.

Such improvement may be performed by adding additives to the saline solution and/or to the desalted water, however such addition needs to be constantly maintained and is costly.

It is an object of the present invention to provide novel filters that require lower pressure to provide a given flux, or provide a greater flux at a given pressure.

Further objects and advantages of this invention will appear as the description proceeds.

### SUMMARY OF THE INVENTION

According to a first aspect, a reverse-osmosis membrane filter is provided, the filter comprising:
a porous support layer;
a porous skin layer, and
at least one water binding composition predominantly bound between the skin layer and the support layer.

In some embodiments the water binding composition comprises at least one peptoid.

In some embodiments in the water binding composition particularly consists of at least one peptoid.

The peptoid is for example a N-substituted glycine peptoid.

In some embodiments the peptoid is selected from a peptoid group consisting of Ac(Nser), Ac(Nme)₃, and mixtures thereof.

The skin layer is typically selected from a group consisting of: polyamides, cellulose acetates, polyimides, polybenzimidazole and mixtures thereof.

In some preferred embodiments the skin layer comprises polyamides and the peptoid is selected from a group consisting of Ac(Nser), Ac(Nme)₃, and mixtures thereof, and

the peptoid is bound to the skin layer.

The support layer in some embodiments comprises polysulfone.

In some embodiments the peptoid is bound to the support layer.

In preferred embodiments the porous skin layer is capable of rejecting ions and small molecules, deposited on the support layer.

According to another aspect, a method of producing an improved reverse osmosis filter is provided, the method comprising:
providing a porous support layer;
providing a porous skin layer;
binding at least one peptoid to the skin layer, and
depositing the skin layer on the support layer.

In some method embodiments the skin layer comprises a composition selected from a group consisting of: polyamides, cellulose acetates, polyimides, polybenzimidazole and mixtures thereof, and the method
further comprises coupling the at least one peptoid to the skin layer with coupling agent selected from a group consisting of: a peptoid-amine coupling agent, a peptoid-cellulose acetate coupling agent, and a peptoid-imide coupling agent and mixtures thereof.

In some embodiments the peptoid-amine coupling agent is a carboxyl activating agent capable of coupling primary amines to the carboxyl.

In some particular embodiments the coupling agent is EDC.

According to another aspect, a reverse-osmosis membrane filter is provided, the filter comprising:
a porous support layer;
a porous skin layer, and
at least one water binding composition predominantly bound between the skin layer and the support layer.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the specification, including definitions, will control. In addition, the materials, methods, and examples are illustrative only and not intended to be limiting.

### DESCRIPTION OF THE PREFERRED EMBODIMENTS

Before explaining at least one embodiment in detail, it is to be understood that the invention is not limited in its application to the details of construction and the arrangement of the components set forth in the following description. The invention is capable of other embodiments or of being practiced or carried out in various ways. Also, it is to be understood that the phraseology and terminology employed herein is for the purpose of description and should not be regarded as limiting.

In WO2011154946 a purification unit is described, that is named a “forward osmosis purification unit”. The unit includes an inlet chamber into which an unpurified feed solution is introducible, an outlet chamber, and a dual membrane section. The dual membrane section includes a first semi-permeable membrane in fluid communication with the inlet chamber, a second semi-permeable membrane in fluid communication with the outlet chamber, a plurality of expandable cells interposed between the first and second membranes, and a draw solution of an osmotic pressure significantly greater than the osmotic pressure of the feed solution. According to WO2011154946 a sufficient amount of solvent is permeable through the first membrane to increase the hydraulic pressure of the draw solution within the cells, while solutes of the feed solution are substantially rejectable. WO2011154946 further states that the hydraulic pressure of the draw solution is sufficiently high to force the permeate to be discharged from the second membrane to the outlet chamber while the draw agent is substantially rejected.

Modifying and/or adding solutions is the major way facilitation of filtration is achieved. The present approach creates an effect similar to forward osmosis, however no solvents are required to be added to the solutions subject to filtration (or to the filtered solution), in order to improve the filtration, which simplifies the filtration and reduces its cost.

When desalinating saline water by passing the saline water through a membrane, the presence of water binding molecules (WBM) in a side of the membrane facing the saline solution could virtually increase the concentration of the desalted water in the vicinity of this side. This virtual solution state reduces the difference in free energy between the two sides of the membrane (between the saline solution and the desalted water). Thus, lower external pressure is required for the process, making the desalination processes more energy favorable.

The inventors of the present invention have found that water binding molecules (WBM) indeed can be used for reducing the free enthalpy of the filtered water and thus, the applied external pressure that is required for the process will be lower.

According to one aspect, an improved reverse-osmosis membrane filter is provided. The membrane comprises:
a porous support layer;
a porous skin layer, capable of rejecting ions and small molecules, deposited on the support layer, and
a least one peptoid predominantly bound (to the skin layer and/or support layer) between the skin layer and the support layer.

Peptoids are molecules that bridge synthetic polymers and biological polymers. These molecules present high chemical stability and low toxicity; thus they are suitable for a variety of applications. The peptoid structure is shown below, and the much more commonly known peptide structure is depicted alongside for comparison sake.

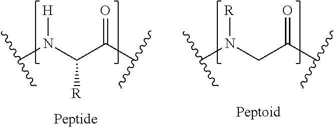

N-substituted glycine peptoids particularly stand out as a family of peptidomimetic oligomers that may have a good affinity to water molecules. Peptoids can be synthesized with precise control over the sequence of highly diverse side chain functional groups, enabling a robust investigation of structure-property relationships. Huang et al. [PNAS Vol. 109, no. 49, pp. 19922-19927] demonstrated that specific peptoids with carboxylic end groups and side chains bearing hydroxyl (Ac(Nser)3), or ether (Ac(Nme)3), indicated below, reduce the freezing point of water much more than it is expected from their colligative effects alone.

The inventors realized that the reduction of freezing point phenomenon may indicate that these molecules form very strong chemical bonds with the water molecules and thus significantly reduce the water enthalpy of the filtered water, and in effect reduce the energy required for filtration. As a starting point the inventors set out to attempt to attach these peptoids to membrane filters, not on the side intended to be exposed to the saline solution.

### Example 1

### “Wet” Preparation of Ac(Sar)3 Peptoid

### Step #1: Preparation of Trifluoroacetamidoethanol

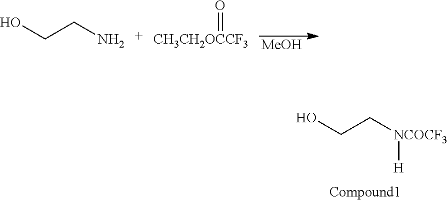

To a solution of 2-aminoethanol (20 gr, 0.32 moles) in methanol (50 mL) a solution of ethyltrifluoroacetate (50 gr, 0.35 moles) in methanol (50 mL) was added dropwise at room temperature with stirring.

The reaction mixture was stirred for 18 hours, followed by evaporating to dryness, to obtain a white solid. The product Compound 1 was used for the next step without purification.

### Step #2: Preparation of 2-trityltrifluoroacetamidoethanol

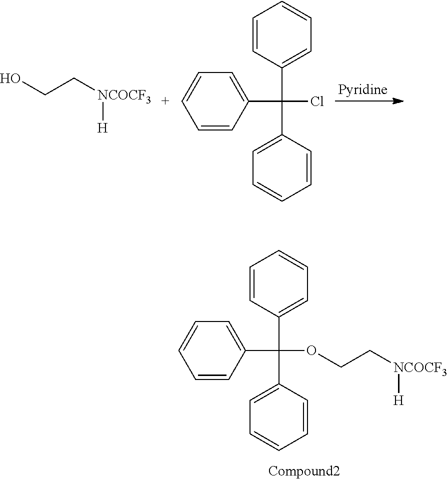

To a solution of trifluoroacetamidoethanol (15.7 gr, 100 millimoles) in dry pyridine (50 mL), one portion tritylchloride (30 gr, 107 millimoles) was added. The reaction mixture was stirred for 18 hours at room temperature, followed by addition of methanol (20 mL) while stirring for 20 minutes. The reaction mixture was evaporated to dryness, to obtain a white solid. The product Compound 2 was used for the next step without purification.

### Step #3: Preparation of 2-trityl aminoethanol

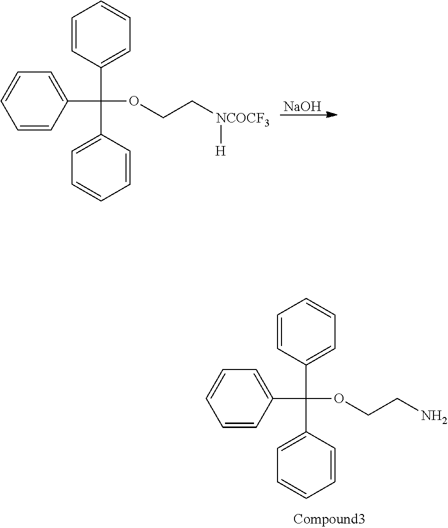

To a solution of Compound 2 in methanol (100 mL), a solution of 2N sodium hydroxide (50 mL) was added. The reaction mixture was stirred for 3 hours at room temperature, followed evaporation to dryness. The solid product was extracted with ethylacetate (200 mL), followed by washing with brine, and the organic solution was dried over anhydrous sodium sulfate. The ethyl acetate was evaporated to dryness to obtain a white solid, which gives a positive ninhydrin test. The product was purified on a silica gel column using a solution of (5 methanol:95 ethylacetate). A white solid was obtained.

Rf: 0.23 (5 methanol:95 ethylacetate).

Yield from the three steps was 73%.

### Step #4: Reaction of Compound 3 with 2-bromoacetamide

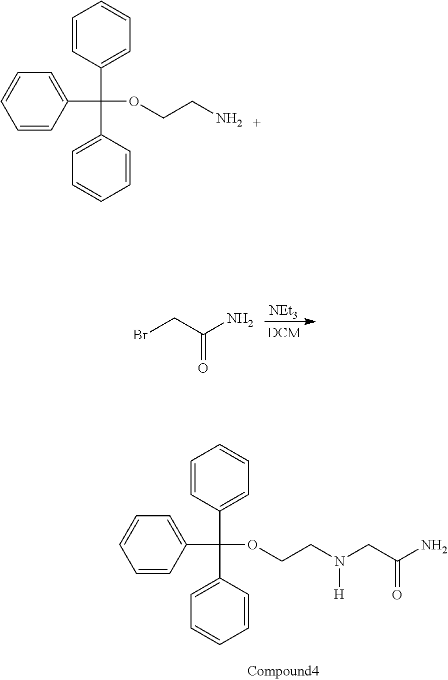 [SMILES: NCCOC(c1ccccc1)(c1ccccc1)c1ccccc1 ; NC(=O)CBr ; NC(=O)CNCCOC(c1ccccc1)(c1ccccc1)c1ccccc1 ; "NEt3"]

To a stirred solution of Compound 3 (4.34 gr, 14.3 millimoles) in dry dichloromethane (100 mL) (DCM), and triethylamine (10 gr, 98 millimoles), 2-bromoacetamide (1.97 gr, 14.3 millimoles) was added as a solid in portions over a period of 1 hour at room temperature. The reaction mixture was stirred for 18 hours at room temperature, followed by evaporation to dryness. The product was extracted with ethyl acetate (200 mL), followed by washing with brine, and the organic solution was dried over anhydrous sodium sulfate. The ethyl acetate was evaporated to dryness to obtain a white solid. The product was purified on silica gel column using a gradient of ethyl acetate to (10 methanol:90 ethylacetate). A white solid was obtained.

Rf: 0.42 (10 methanol:90 ethylacetate).

Yield: 4.2 gr, 81.5%.

### Step #5: Reaction of Compound 4 with 2-bromoecetic acid

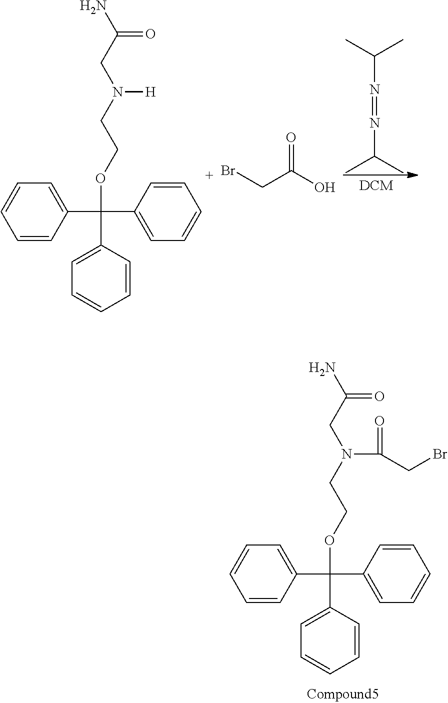

To a solution of Compound 4 (0.75 gr, 2 millimoles) in dry DCM (50 mL), 2-bromoecetic acid (0.31 gr, 2.2 millimoles) was added in one portion. To this solution a solution of diisopropylcarbo diimide (3504) in DCM (10 mL) was added dropwise at room temperature. The reaction mixture was stirred for 5 hours, followed by evaporation to dryness. The product was extracted with ethyl acetate (100 mL), followed by washing with brine, and the organic solution was dried over anhydrous sodium sulfate. The ethyl acetate was evaporated to dryness to obtain a white solid. The product was purified on silica gel column using a gradient of DCM to (10 methanol:90 ethylacetate). A white solid was obtained.

Rf: 0.71 (10 methanol:90 ethylacetate).

Yield: 091 gr, 91%.

### Step #6: Reaction of Compound 5 with Compound 3

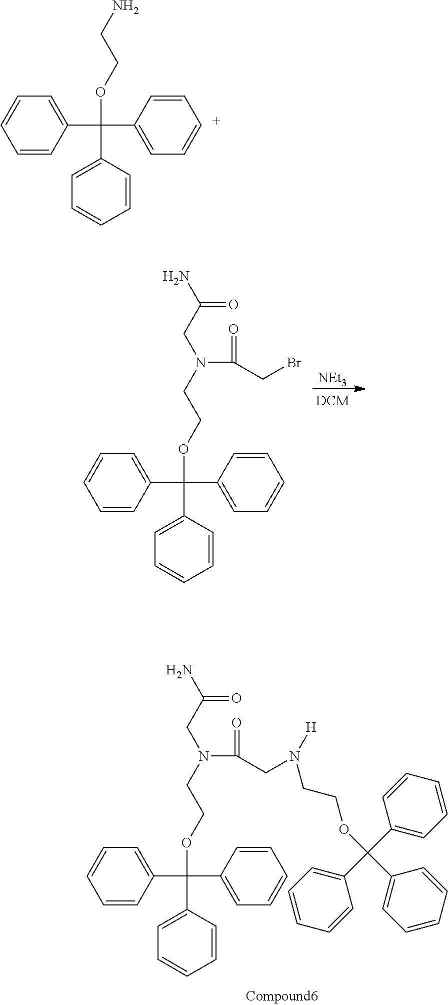 [SMILES: NCCOC(C1=CC=[C+]C=C1)(c1ccccc1)c1ccccc1 ; NC(=O)CN(CCOC(c1ccccc1)(c1ccccc1)c1ccccc1)C(=O)CBr ; NC(=O)CN(CCOC(c1ccccc1)(c1ccccc1)c1ccccc1)C(=O)CNCCOC(c1ccccc1)(c1ccccc1)c1ccccc1 ; "NEt3"]

To a stirred solution of Compound 3 (1.0 gr, 3.3 millimoles) in dry dichloromethane (100 mL) (DCM), and triethylamine (10 gr, 98 millimoles), Compound 5 (1.0 gr, 2.07 millimoles) was added as a solid in portions over a period of 1 hour at room temperature. The reaction mixture was stirred for 18 hours at room temperature, followed by evaporation to dryness. The product was extracted with ethyl acetate (200 mL), followed by washing with brine, and the organic solution was dried over anhydrous sodium sulfate. The ethyl acetate was evaporated to dryness to obtain a white solid. The product was purified on silica gel column using a gradient of ethyl acetate to (5 methanol:95 ethylacetate). A white solid was obtained.

Rf: 0.47 (5 methanol:95 ethylacetate).

Yield: 1.6 gr, 68.6%.

### Step #7: Reaction of Compound 6 with 2-bromoacetic acid

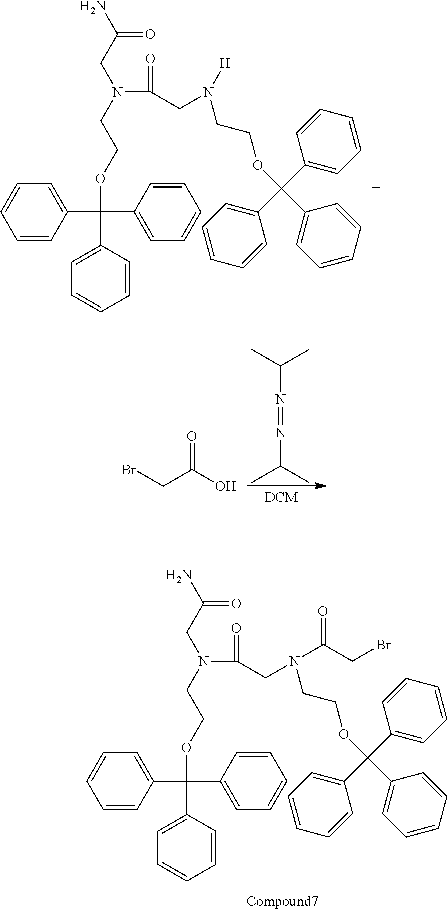

To a solution of Compound 6 (2.41 gr, 3.42 millimoles) in dry DCM (50 mL), 2-bromoecetic acid (0.55 gr, 3.95 millimoles) was added in one portion. To this solution a solution of diisopropylcarbo diimide (530 μL, 3.78 millimoles) in DCM (10 mL) was added dropwise at room temperature.

The reaction mixture was stirred for 5 hours, followed by evaporation to dryness. The product was extracted with ethyl acetate (100 mL), followed by washing with brine, and the organic solution was dried over anhydrous sodium sulfate. The ethyl acetate was evaporated to dryness to obtain a white foam solid.

Rf: 0.77 (5 methanol:95 ethylacetate).

Yield: 2.71 gr, 96%.

The product (Compound 7) was used without further purification.

### Step #8: Reaction of Compound 7 with Ethanolamine

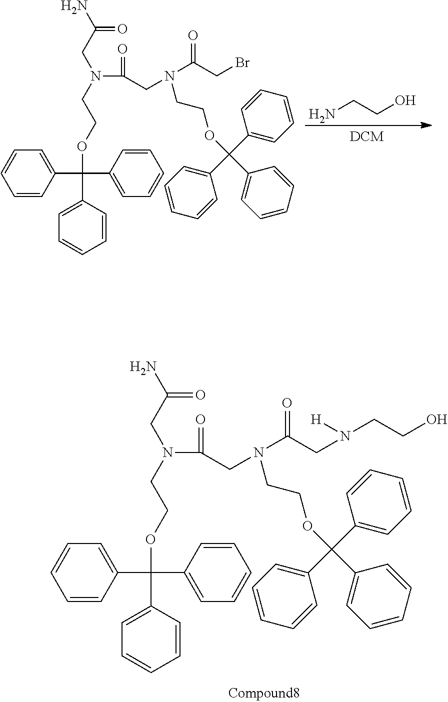

To a solution of Compound 7 which was obtained from the previous step, in DCM (50 mL), aminoethanol (5 mL) and triethylamine (5 mL) were added. The reaction mixture was stirred for 18 hours at room temperature, followed by evaporation to dryness. The product was extracted with ethyl acetate (100 mL), followed by washing with brine, and the organic solution was dried over anhydrous sodium sulfate. The ethyl acetate was evaporated to dryness to obtain a white foam solid.

Rf: 0.26 (10 methanol:90 dichloromethane).

Yield: 1.73 gr, 84%.

### Step #9: Reaction of Compound 8 with Succinic Anhydride

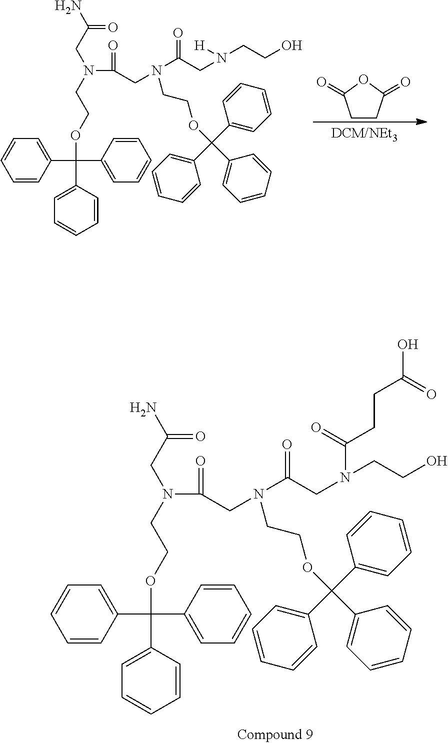 [SMILES: NC(=O)CN(CCOC(c1ccccc1)(c1ccccc1)c1ccccc1)C(=O)CN(CCOC(c1ccccc1)(c1ccccc1)c1ccccc1)C(=O)CNCCO ; O=C1CCC(=O)O1 ; NC(=O)CN(CCOC(c1ccccc1)(c1ccccc1)c1ccccc1)C(=O)CN(CCOC(c1ccccc1)(c1ccccc1)c1ccccc1)C(=O)CN(CCO)C(=O)CCC(=O)O]

To a solution of Compound 8 (2 gr, 2.48 mmoles) in dry DCM (30 mL), and in triethylamine (3 mL), succinic anhydride (1 gr, 10 millimoles) was added in one portion. The reaction was stirred for 18 hours at room temperature, followed by evaporation to dryness. The product was extracted with ethyl acetate (100 mL), followed by washing with brine, and the organic solution was dried over anhydrous sodium sulfate. The ethylacetate was evaporated to dryness to obtain a white solid.

The product was used for the next step without further purification.

### Step #10: Reaction of Compound 9 with Acetic Acid

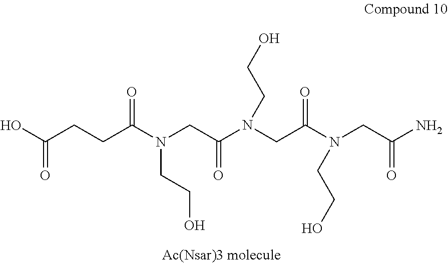 [SMILES: NC(=O)CN(CCO)C(=O)CN(CCO)C(=O)CN(CCO)C(=O)CCC(=O)O]

To the product obtained from the previous step a solution of 80% acetic acid in water (30 mL) was added. The reaction mixture was refluxed for 1 hour followed by evaporation to dryness. The crude product was purified on silica gel column using a gradient of ethyl acetate to (15 methanol:85 DCM). A white solid was obtained.

### Example 2

### “Solid” Preparation of Ac(Sar)₃ Peptoid

Solid-phase synthesis of peptoid oligomers was performed in fritted syringes on a Rink amide resin. 100 mg of resin with a loading level of 0.82 mmol·g−1 was swollen in 4 mL of dichloromethane (DCM) for 40 min. Following swelling, the Fmoc protecting group was removed by treatment with 2 mL of 20% piperidine in dimethylformamide (DMF) for 20 min. After de-protection and after each subsequent synthetic step, the resin was washed three times with 2 mL of DMF, one minute per wash.

Peptoid synthesis was carried out with alternating bromoacylation and amine displacement steps. For each bromoacylation step, 20 equiv bromoacetic acid (1.2 M in DMF, 8.5 mL g−1 resin) and 24 equiv N,N′-diisopropylcarbodiimide (neat, 2 mL g−1 resin) were added to the resin, and the mixture was agitated for 20 min.

After washing, 20 eq. of the required amine (1.0 M in DMF) were added to the resin and agitated for 20 min. For desired sequence we used O-tert-butyl-dimethylsilyl-2-ethanolamine and for last acylation step was used succinic acid instead of bromoacetic acid.

When the desired sequence was achieved, the peptoid products were cleaved from the resin by treatment with 95% trifluoroacetic acid (TFA) in water (50 mL g−1 resin) for 30 minutes.

After filtration, the cleavage mixture was concentrated by rotary evaporation under reduced pressure for large volumes or under a stream of nitrogen gas for volumes less than 1 mL.

Cleaved samples were then re-suspended in 50% acetonitrile in water and lyophilized to powders.

Peptoids were purified by preparative High performance liquid chromatography (HPLC) using a C18 column. Products were detected by UV absorbance at 230 nm during a linear gradient conducted from 5% to 95% solvent B (0.1% TFA in HPLC grade acetonitrile) over solvent A (0.1% TFA in HPLC grade water) in 50 minutes with a flow rate of 5 mL min-1. MS (ESI): m/z=420.4 calculated for C16H28N4O9 [M]+. found: 422.1 (Advion expression CMS).

### Example 3

### Modifying a Membrane with a Bound Peptoid

Common membrane polymers that are used for the manufacturing of membranes applicable to water treatment are: cellulose acetate or nitrate, polyamide, polycarbonate, polysulfone and polyethersulfone, polypropylene, polyvinylidene fluoride—each resulting in different membrane properties. Thin-film composite membranes (TFC) with a polyamide top layer are the most common reverse osmosis membranes used today for desalination (process that remove salt and other minerals from saline water) and thus these membranes were an selected as a starting point for membrane modification.

The polyamide layer of these membranes is usually a skin of 100-200 nm thickness, which is formed on top of a ˜150 μm thick microporous polysulfone support, by interfacial polymerization. The polyamide layer manufacture based on a polycondensation reaction between two monomers meta-phenylene diamine and trimesoyl chloride (TMC):

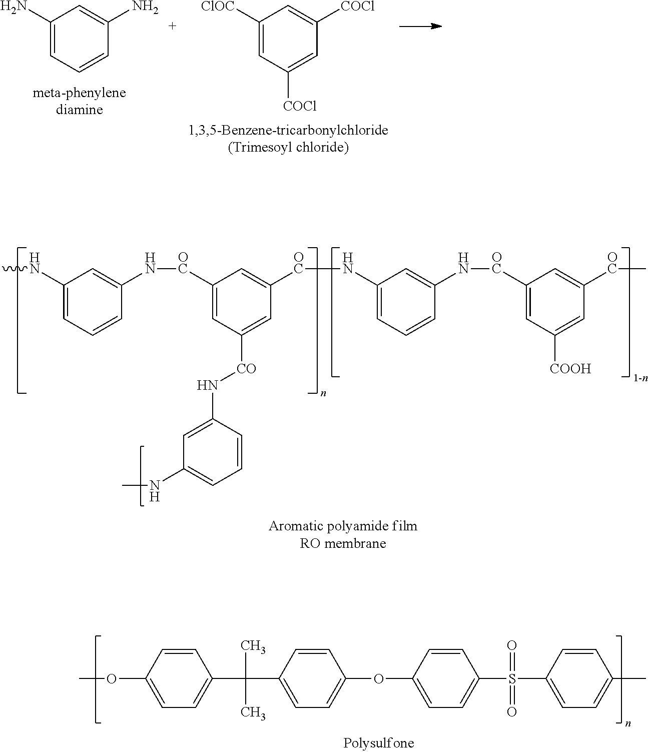

There are no known chemical bonds between the polysulfone layer and the polyamide layer. Rather, the polyamide adheres to the polysulfone support by physical bonds.

As a first approach to improving membranes by incorporation of peptoids, the WBM is attached to membrane in the polyamide-polysulfone interface. WBM could be inserted into a flat sheet commercial membrane from the polysulfone side and bind to the polyamide internal layer.

For example, Ac(Nser)₃ molecules (WBM) could theoretically bond to excess amino groups which are said to exist in the polyamide interior layer.

Experiments were conducted to bind peptoids with existing polyamide films by means of reactions with coupling agents known to aid in peptide synthesis:

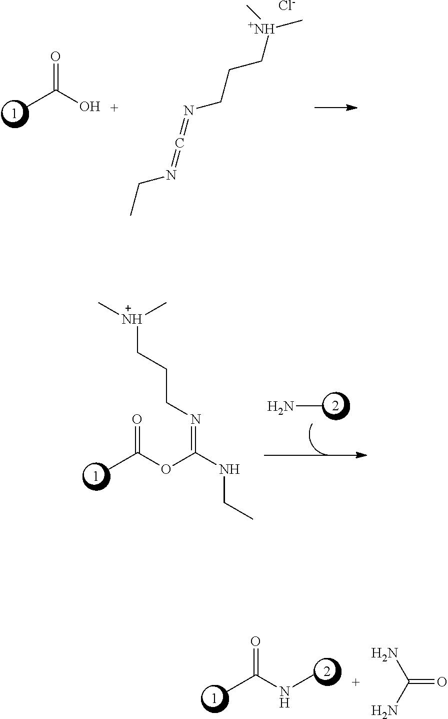

In this reaction a carboxylic acid of the peptoid reacts with a coupling agent (EDC in the scheme above) to form an active acylurea, which is then reacted with free amine groups in the polyamide membrane.

The successful formation of a modified membrane by using EDC was surprising considering that other coupling agents DIC, DMF and DCM all ruined the membrane or produced esters in repeat experiments under various ratios of the reagents and various conditions.

In general, at present the preferred coupling agents are carboxyl activating agents that can couple the carboxyl to primary amines to yield amide bonds.

In order to prevent reaction between the peptoid and carboxylic groups on the skin the reaction was performed in special cells that contained 6 mL water, the filter, the peptoid and the linker. The cells allowed diffusion only to the interface between the polysulfone support and the polyamide skin, and physically prevented access of the peptoid and coupling agent to the side of the polyamide skin facing away from the polysulfone support.

Control cells contained the same setup but without the peptoids.

The filters were left immersed in the cells for several hours to allow diffusion of the peptoid and the EDC though the polysulfone layer to the interface between the support and the skin.

### Example 4

### Tests on the Modified Membrane

The permeability and the salt rejection of the modified membranes prepared as described in Example 2 were measured using a cross flow filtration setup. The feed was deionized water.

The whole setup was cleaned using bleach followed by a solution of EDTA, and then rinsed with deionized water for about 5 times and then the experiment was conducted. Control membranes were provided as described in Example 2 but without the peptoid. The permeability was measured using two different sets of parameters:

1) The system was allowed to operate for 30 minutes after start, after which the permeate was collected for each pressure for 5 minutes. [40, 50, 60 bar]
2) The system was allowed to operate for 60 minutes after start, after which the permeate was collected for each pressure for 30 minutes. [10, 20 bar]
The Salt rejection was measured using NaCl Salt (2 g/l) at a pressure of 50 bar and flow rate of around 50 lph.
The following data summarize the calculations. Table 1 summarizes the results from three control membranes C1-1, C1-2 and C1-3. Table 2 summarizes results from three modified membranes T1-1, T1-2 and T1-3.

TABLE 1

| Name | P(bar) | Permeability(l/hm²bar) | Rejection |
|---|---|---|---|
| C1-1 | 40 | 0.44 | 98.7 |
| C1-1 | 50 | 0.49 | |
| C1-1 | 60 | 0.50 | |
| C1-2 | 40 | 0.44 | 98.4 |
| C1-2 | 50 | 0.51 | |
| C1-2 | 60 | 0.56 | |
| C1-3 | 40 | 0.44 | 98.3 |
| C1-3 | 50 | 0.47 | |
| C1-3 | 60 | 0.55 | |
| C1-3 a | 10 | 0.43 | |
| C1-3 a | 20 | 0.46 | |

TABLE 2

| Name | P(bar) | Permeability (l/hm2bar) | Rejection (%) |
|---|---|---|---|
| T1-1 | 40 | 0.54 | 98.4 |
| T1-1 | 50 | 0.60 | |
| T1-1 | 60 | 0.69 | |
| T1-2 | 40 | 0.51 | 98.2 |
| T1-2 | 50 | 0.62 | |
| T1-2 | 60 | 0.69 | |
| T1-3 | 40 | 0.56 | 98.52 |
| T1-3 | 50 | 0.64 | |
| T1-3 | 60 | 0.67 | |
| T1-3 | 10 | 0.47 | 98.2 |
| T1-3 | 20 | 0.48 | |
| T1-1 a | 10 | 0.51 | |
| T1-1 a | 20 | 0.52 | |
| T1-2 a | 10 | 0.56 | |
| T1-2 a | 20 | 0.65 | |

The results demonstrate significantly improved permeability at various pressures, without adverse effect on the rejection.

Similar positive results in comparison to the control filters were obtained from subjecting the test and control filters to a dead-end filtration setup.

The improved membranes' performance may translate into a reduction in energy consumption of about 10-30% in the filtration process.

After the filtration tests described above were completed the presence of the peptoid in the filter was supported by indication of the peptoid functional groups in IR spectroscopy analysis results obtained from subjecting the polyamide skin to such analysis.

In the examples above the peptoid is bound to a ready-made filter and may thus modify commercial filters and filters already put in use.

Alternatively, these water binding molecules are incorporated into the membrane during the manufacture process. The WBM is attached to the diamine groups and intrudes into the polyamide-polysulfone interface during an interfacial polymerization procedure.

### Example 5

### Interfacial Polymerization (IP) Procedure

The membrane-forming system includes m-phenylendiamine (MPD) in water, and TMC in hexane or heptanes.

The IP films are supported on microporous polysulfone films. Unsupported polyamide films are prepared by carefully adding a TMC solution within 1 to 2 s to an aqueous MPD solution.

In some embodiments the MPD solution comprises at least one peptoid; in other embodiments the TMC solution comprises the peptoid.

In some embodiments the MPD solution comprises at least one peptoid-MPD coupling agent; in other embodiments the TMC solution comprises the coupling agent.

Composite membranes are prepared immersing the polysulfone support in an aqueous solution of MPD. After removal of excess MPD solution from the surface of the support, the wet film is immediately covered with TMC in organic solution and then dried. The composite membrane is extracted in hot distilled water 50-60° C.

In some embodiments the peptoid is a N-substituted glycine peptoid.

In some embodiments peptoid is selected from a group consisting of Ac(Nser), Ac(Nme)3, and mixtures thereof.

In some preferred embodiments the peptoids comprise a short chain length and a small bonding group such as carboxyl.

In some embodiments the skin layer comprises a composition selected from a group consisting of: polyamides, cellulose acetates, polyimides, polybenzimidazole and mixtures thereof.

In another aspect the peptoid is bound to the support layer.

In another aspect a method of producing an improved reverse osmosis filter is provided. The method comprises:

providing a porous support layer;

providing a porous skin layer, capable of rejecting ions and small molecules;

binding at least one peptoid to the skin layer, and

depositing the skin layer on the support layer.

The skin layer may comprises a composition selected from a group consisting of: polyamides, cellulose acetates, polyimides, polybenzimidazole and mixtures thereof, and may further comprise coupling the at least one peptoid to the skin layer with coupling agent selected from a group consisting of: a peptoid-amine coupling agent, a peptoid-cellulose acetate coupling agent, a peptoid-imide coupling agent, and a peptoid-benzimidazole coupling agent and mixtures thereof.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed:

1-15. (canceled)

16. A reverse osmosis membrane filter comprising a porous support layer, a porous skin layer capable of rejecting ions and small molecules and at least one peptoid predominantly bound to the skin layer and/or support layer, between the skin layer and the support layer.

17. The reverse osmosis membrane filter of claim 16 wherein said peptoid is bound to the skin layer deposited on the support layer and is predominantly bound between the skin layer and the support layer.

18. The skin layer of claim 16 wherein comprising at least one component selected from the group consisting of polyamides, cellulose acetates, polyimides, polybenzimidazoles and mixtures thereof.

19. The reverse osmosis membrane filter of claim 16, wherein the peptoid bound to the skin layer is a N-substituted glycine peptoid.

20. The reverse osmosis membrane filter of claim 19, wherein the N-substituted glycine peptoid is selected from the group consisting of di-glycinamide ketone, tri-N′,N″,N′″-hydroxyethyl, N′″-carboxyoxopropyl (Ac(Nser)3), di-glycinamide ketone, tri-N′,N″,N′″-methylethylether, N′″-carboxyoxopropyl (Ac(Nme)3) and mixtures thereof.

21. The reverse osmosis membrane filter of claim 19, wherein the skin layer comprises polyamides, the peptoid is selected from the group consisting of Ac(Nser)3, Ac(Nme)3 and mixtures thereof, and the peptoid is bound to the skin layer.

22. The reverse osmosis membrane filter of any of the previous claims, wherein the support layer comprises polysulfone.

23. The reverse osmosis membrane filter of claim 22, wherein the peptoid is bound to the support layer.

24. The reverse osmosis membrane filter of claim 16, wherein the porous skin layer is capable of rejecting ions and small molecules deposited on the support layer.

25. A method of producing an improved reverse osmosis membrane filter comprising providing a porous support layer, providing a porous skin layer, binding at least one peptoid to the skin layer or support layer and depositing said skin layer on the support layer.

26. A method for preparing a peptoid-bound skin layer, comprising coupling at least one peptoid to the skin layer of claim 18 by using a coupling agent selected from the group consisting of a peptoid-amine coupling agent, a peptoid-cellulose acetate coupling agent, a peptoid-imide coupling agent, a peptoid-polybenzimidazole coupling agent and mixtures thereof.

27. The method of claim 26, wherein the peptoid-amine coupling agent is a carboxyl activating agent capable of coupling primary amines to the carboxyl.

28. The method of claim 26 wherein the coupling agent is 1-ethyl-3-(3-dimethylaminopropyl) carbodiimide (EDC).

29. A peptoid-bound membrane skin prepared by the method of claim 26, coupling at least one peptoid to the skin layer by using the coupling agent.

30. The reverse osmosis membrane filter of claim 16, comprising a peptoid-bound skin layer, wherein enabling improved reverse osmosis parameters selected from the group consisting of reduced energy consumption, lower pressure required, higher throughput, improved permeability at various pressures and combinations thereof, as compared to a non-peptoid-bound membrane filter.
